(19) United States
(12) Patent Application Publication
(10) Pub. No.: US 2025/0164403 A1
(43) Pub. Date: May 22, 2025

(54) RAPID DETECTION AND ANALYSIS OF BIOLOGICAL COMPONENTS USING SURFACE ENHANCED RAMAN SPECTROSCOPY

(71) Applicant: VIRGINIA TECH INTELLECTUAL PROPERTIES, INC., Blacksburg, VA (US)

(72) Inventors: Aditya Garg, Blacksburg, VA (US); Wei Zhou, Blacksburg, VA (US); Linsey Marr, Blacksburg, VA (US); Peter Vikesland, Blacksburg, VA (US); Nisha Duggal, Blacksburg, VA (US)

(21) Appl. No.: 18/951,181

(22) Filed: Nov. 18, 2024

Related U.S. Application Data
(60) Provisional application No. 63/600,420, filed on Nov. 17, 2023.

(51) Int. Cl. G01N 21/65; G01N 1/44; G01N 33/543; G01N 33/569; G16B 40/10; G16B 40/20

(52) U.S. Cl. CPC G01N 21/658; G01N 1/44; G01N 33/54346; G01N 33/56983; G16B 40/10; G16B 40/20; G01N 2201/0221; G01N 2201/129; G01N 2333/11; G01N 2333/165; G01N 2333/18

(57) ABSTRACT

The present disclosure is directed to rapid detection and analysis of biological components using surface-enhanced Raman spectroscopy (SERS). One general aspect includes a method for preparing biological components from a biological sample for label-free SERS. The method also includes providing a Raman-background-free surfactant buffer to the biological sample. The method also includes agitating and heating the biological sample in the buffer to accelerate a lysis process thereby decomposing structural components of the biological sample. The structural components are substantially free of Raman background interference and are optimized for SERS signal enhancement.

### CROSS-REFERENCE TO RELATED APPLICATION

This application claims the benefit of and priority to U.S. Provisional Patent Application No. 63/600,420, filed Nov. 17, 2024, entitled “SYSTEMS AND METHODS FOR DETECTING ENVIRONMENTAL VIRAL COMPONENTS,” the contents of which is hereby incorporated herein by reference in its entirety.

### STATEMENT REGARDING FEDERALLY SPONSORED RESEARCH OR DEVELOPMENT

This invention was made with government support under OISE-1545756, CBET-2029911, CBET-2231807, and NNCI 1542100 awarded by the National Science Foundation. The government has certain rights in the invention.

### BACKGROUND

The analysis and detection of biological components, including viruses and microorganisms, in environmental samples is a significant area of focus across multiple scientific and industrial fields. Methods for such analysis often rely on advanced molecular and spectroscopic techniques to characterize biological components with precision. These methods enable the study of structural elements such as nucleic acids, proteins, and other molecular constituents that are integral to understanding biological processes and interactions.

Surface-enhanced Raman spectroscopy (SERS) has become a well-recognized analytical technique for molecular fingerprinting due to its ability to enhance vibrational signals through plasmonic nanostructures. SERS applications span molecular diagnostics, environmental monitoring, and chemical analysis, with its utility arising from its specificity and sensitivity to molecular structures. The development of sample preparation protocols and data analysis techniques has played a critical role in enabling SERS to analyze complex samples effectively.

Environmental matrices, such as air, surface particulates, and fluids, present unique challenges for molecular analysis due to the presence of various interfering substances and the complexity of the sample's composition. These challenges necessitate the continued refinement of methodologies to ensure accurate and reliable detection of biological and molecular components in such settings.

### BRIEF SUMMARY OF THE INVENTION

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

The present disclosure is directed to rapid detection and analysis of biological components using surface-enhanced Raman spectroscopy (SERS). One general aspect includes a method for preparing biological components from a biological sample for label-free surface-enhanced Raman spectroscopy (SERS). The method also includes providing a Raman-background-free surfactant buffer to the biological sample. The method also includes agitating and heating the biological sample in the buffer to accelerate a lysis process thereby decomposing structural components of the biological sample. The structural components are substantially free of Raman background interference and are optimized for SERS signal enhancement.

Implementations may include one or more of the following features. The method may include mixing the structural components with gold, silver, and/or other nanoparticles to form a mixture; and condensing the mixture onto a hydrophobic surface to form enriched analytes with nanogap SERS hotspots. The biological sample may include a virus, a bacterium, a fungus, or a combination thereof. The structural components may include at least one of a protein, a lipid, a carbohydrate, or a nucleic acid. The surfactant buffer may include sodium dodecyl sulfate (SDS) or another Raman-inactive buffer. The biological sample is heated to a temperature between about 35° C. and 60° C. to optimize lysis efficiency while preserving structural component integrity. The agitation is performed using ultrasonic sonication at a frequency between 20 kHz and 40 kHz. The method may include enriching the structural components within a compact detection area by co-condensing the lysed sample and nanoparticles on a silane-functionalized hydrophobic surface.

One general aspect includes a method for continuous, real-time monitoring of microorganisms in environmental samples. The method includes collecting a sample containing microorganisms from air, surfaces, or fluids. The method also includes lysing the microorganisms into structural components using a Raman-background-free surfactant buffer, agitation, and controlled heating. The method also includes mixing the structural components with gold, silver, and/or other nanoparticles on a hydrophobic surface to create analytes with nanogap SERS hotspots. The method also includes enriching the analytes within a compact detection area through co-condensation. The method also includes capturing label-free SERS spectra of the structural components from the enriched analytes. The method also includes analyzing the captured spectra using a machine learning model trained on a library of SERS spectral profiles.

Implementations may include one or more of the following features. The method where the machine learning model is trained on a library may include spectral profiles of one or more macromolecular components associated with microorganisms. The library may include SERS data for structural components of SARS-Cov-2, influenza a virus, and zika virus. The environmental sample is collected from an air filter, a high-touch surface, or a fluid sample. The method may include generating real-time alerts based at least in part on a detected presence of a target microorganism. The hydrophobic surface may include silane-treated aluminum foil. The co-condensation is performed at a temperature between about 20° C. and 30° C. to preserve a structural integrity of the sample.

One general aspect includes a portable system for label-free detection of biological components in environmental samples. The portable system also includes a lysis unit configured to process biological samples using a surfactant buffer, agitation, and controlled heating. The system also includes a nanoparticle aggregation module configured to mix lysate with gold, silver, and/or other nanoparticles to form a mixture and condense the mixture onto a hydrophobic surface to create analytes with nanogap SERS hotspots. The system also includes a Raman spectrometer configured to capture spectral data from the analytes. The system also includes a machine learning module trained with a library of SERS spectral profiles for real-time classification of microorganisms.

Implementations may include one or more of the following features. The system where the lysis unit may include an ultrasonic sonicator capable of operating at frequencies between 20 kHz and 40 kHz. The machine learning module is configured to identify microorganisms based on spectral differences in RNA nucleotide and protein peak compositions. The hydrophobic surface may include a microfluidic channel coated with silane-treated materials. The Raman spectrometer is a compact, portable device optimized for field deployment.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 depicts an operating environment in which a surface-enhanced Raman spectroscopy (SERS)-based biological detection and analysis system may be deployed according to an example implementation.

FIG. 2 depicts a functional workflow of the system of FIG. 1, detailing a transformation of a biological sample into actionable output according to an example implementation.

FIG. 3 depicts a method for preparing biological components from a biological sample for label-free SERS according to an example implementation.

FIG. 4 depicts a method for continuous, real-time monitoring of microorganisms in environmental samples using label-free SERS according to an example implementation.

### DETAILED DESCRIPTION

Traditional detection methods, such as polymerase chain reaction (PCR) and antigen-based assays, often require extensive sample preparation, specialized reagents, and laboratory-based infrastructure, making them less suitable for real-time, field-deployable applications. These limitations underscore the need for a portable, label-free system capable of rapidly detecting microorganisms with high sensitivity and specificity in diverse environmental settings

The present disclosure provides a method for detecting and analyzing biological components using surface-enhanced Raman spectroscopy (SERS). The method employs a novel lysis protocol combining a Raman-background-free surfactant buffer, controlled heating, and agitation techniques to break down biological samples into structural components, such as at least one of a protein, a lipid, a carbohydrate, or a nucleic acid. Unlike conventional lysis protocols, the surfactant buffer eliminates interference from Raman-active reagents, ensuring that only the structural components contribute to the spectral signature. For example, sodium dodecyl sulfate (SDS) allows efficient lysis while maintaining analyte integrity, even for sensitive viral components such as SARS-CoV-2 spike proteins.

Additionally, the present disclosure describes a portable system integrating sample lysis, nanoparticle aggregation, and spectral analysis in a compact design suited for field deployment. A machine learning model, trained on a comprehensive SERS spectral library, is incorporated to accurately identify target microorganisms based on RNA and protein signatures. The modular design of the system enables its use with various sample types, including air particulates, fluid samples, and residues from high-touch surfaces.

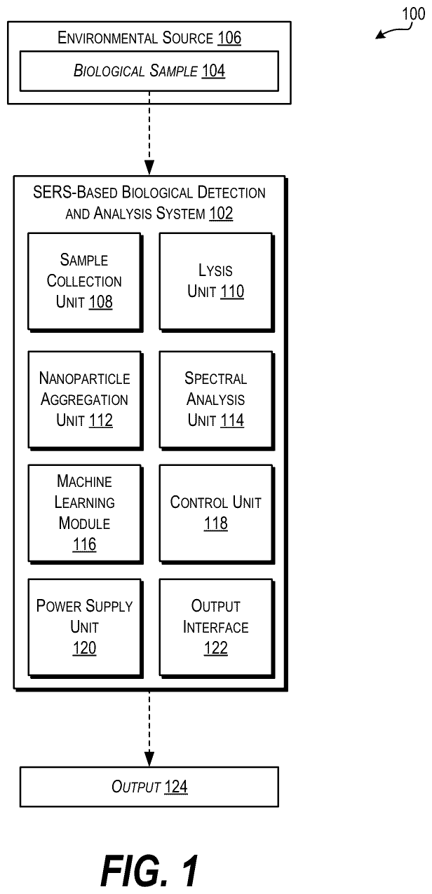

FIG. 1 depicts an operating environment 100 in which a SERS-based biological detection and analysis system 102 (hereafter “the system 102) may be deployed according to an example implementation. Generally, the system 102 is configured to detect and analyze biological components, such as RNA and proteins, from a biological sample 104 collected from an environmental source 106. The system 102 is highly adaptable, allowing the analysis of microorganisms, including viruses like SARS-CoV-2, influenza A, and Zika, bacteria, and other biological entities present in environmental samples such as air, fluids, or surfaces.

The environmental source 106 represents the origin of the biological sample 104, which may include indoor air from heating, ventilation, and air conditioning (HVAC) systems, surface particulates from high-touch surfaces, or water from drinking or wastewater systems. The biological sample 104 includes microorganisms such as viruses, bacteria, or fungi, along with structural components such as one or more proteins (e.g., spike, envelope, and nucleocapsid proteins in SARS-CoV-2), one or more lipids, one or more carbohydrates, one or more nucleic acids (e.g., RNA), or any combination thereof. The biological sample 104 is transferred into the system 102 for comprehensive analysis.

In one or more implementations, the system 102 is a compact, portable system that functions as a highly integrated analytical platform, similar to or as a “laboratory-on-a-chip.” The system 102 combines advanced sample processing, nanoparticle-based signal enhancement, and machine learning-driven spectral analysis into a single device. The system 102 can provide real-time and on-site detection of microorganisms in biological samples 104, the system 102 provides capabilities typically found in sophisticated laboratory setups, including sample lysis, molecular fingerprinting, and data-driven classification, within a modular and field-deployable design.

The system 102 can include multiple interconnected units, each performing specific tasks that enable the detection and characterization of biological components. In the illustrated implementation, these units include a sample collection unit 108, a lysis unit 110, a nanoparticle aggregation unit 112, a spectral analysis unit 114, a machine learning module 116, a control unit 118, a power supply unit 120, and an output interface 122. Together, these units work sequentially to collect, process, analyze, and output actionable information regarding the biological sample 104. The system 102 can be designed for applications in diverse environmental settings, including air, water, and surface contamination analysis.

The sample collection unit 108 initiates the process by acquiring the biological sample 104 from the environmental source 106. The lysis unit 110 then processes the biological sample 104 by breaking down cellular or viral structures into their structural components, such as RNA and proteins, for further analysis. These structural components are passed to the nanoparticle aggregation unit 112, where they are mixed with gold and/or other nanoparticles to create plasmonic hotspots that enhance Raman signal detection. The enhanced analyte is then analyzed by the spectral analysis unit 114, which captures Raman signals corresponding to the molecular fingerprint of the biological sample 104. The data generated by the spectral analysis unit 114 is processed by the machine learning module 116, which uses advanced algorithms and a spectral library to classify and identify microorganisms present in the biological sample 104. Operations of the system 102 are managed by the control unit 118, which synchronizes the components and ensures smooth operation. The system 102 is powered by the power supply unit 120 and delivers results via the output interface 122, which provides real-time feedback and facilitates connectivity with external systems. The output 124 includes qualitative and quantitative information about the biological sample 104, such as microorganism identification and concentration levels.

More particularly, the sample collection unit 108 is designed to collect and prepare the biological sample 104 for analysis. The sample collection unit 108 is capable of handling various sample types, including airborne particulates, fluid samples, and residues collected from surfaces. For air sampling, the sample collection unit 108 can use a filtration mechanism, such as HVAC filters or cyclone separators, to trap particulates containing microorganisms. The sample collection unit 108 can collect fluid samples using pipettes, syringes, or reservoirs designed for temporary storage. The sample collection unit 108 can collect surface residues using swabs or wipe-based mechanisms. The sample collection unit 108 ensures the consistent and contamination-free transfer of biological samples 104 to the lysis unit 110.

The lysis unit 110 processes the collected biological sample 104 by breaking down cellular or viral structures to release intracellular components such as RNA, DNA, and proteins. The lysis unit 110 can include a buffer injection system, which delivers a Raman-background-free surfactant buffer, such as sodium dodecyl sulfate (SDS), to minimize interference in downstream Raman analysis. The lysis unit 110 can also include a heating mechanism, which maintains the biological sample 104 at a controlled temperature, typically between about 35° C. and 60° C., to optimize lysis efficiency. An agitation mechanism, which can employ ultrasonic sonication or mechanical agitation, disrupts membranes and other structural barriers, ensuring the complete release of analyzable components from the biological sample 104. For example, in the case of SARS-CoV-2, the lysis unit 110 breaks down the virus into its structural proteins, such as spike (S), envelope (E), and nucleocapsid (N) proteins, as well as RNA fragments.

The nanoparticle aggregation unit 112 mixes the lysate with gold and/or other nanoparticles to create analytes enriched with nanogap plasmonic hotspots. The nanoparticle aggregation unit 112 can include a nanoparticle reservoir, which stores nanoparticles in a stable state and dispenses the nanoparticles into the lysate via a mixing chamber. The lysate and nanoparticles interact to form plasmonic aggregates, which are then deposited onto a hydrophobic surface, such as silane-treated aluminum foil or the like. The hydrophobic surface promotes condensation and the formation of hotspots that amplify the Raman signals of the structural components of the biological sample 104. For instance, RNA nucleotide bases and viral proteins, such as those from SARS-CoV-2 or Zika virus, interact with the nanoparticles to enhance signal detection.

The spectral analysis unit 114 captures the Raman signals generated from the analytes formed in the nanoparticle aggregation unit 112. The spectral analysis unit 114 includes an excitation laser, typically operating at a wavelength of 785 nanometers (nm) which excites the analytes to produce Raman scattering. A detector captures the scattered photons, generating spectral data that corresponds to the molecular vibrations of the structural components of the biological sample 104. The spectral analysis unit 114 can also incorporate one or more optical filters to minimize background noise and isolate relevant signals. The spectral data produced by the spectral analysis unit 114 is a unique molecular fingerprint of the biological sample 104, enabling precise characterization.

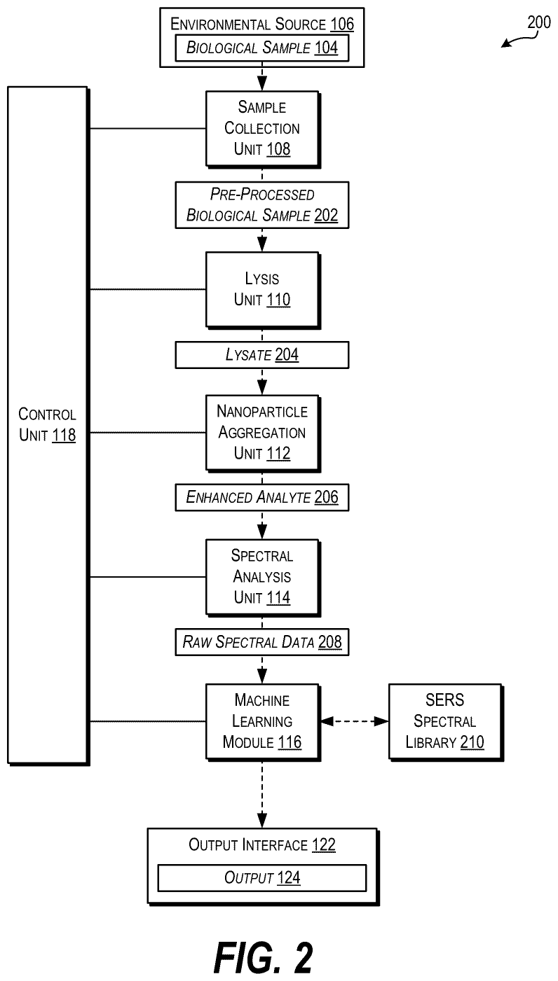

The machine learning module 116 processes the spectral data generated by the spectral analysis unit 114. The machine learning module 116 includes or is otherwise in communication with a SERS spectral library (shown in FIG. 2), which contains profiles of known microorganisms, including SARS-CoV-2, influenza A, and Zika, and their constituent lipids, nucleic acids, proteins, and carbohydrates. The machine learning module 116 can incorporate real-time updates to the SERS spectral library, enabling rapid adaptation to emerging pathogens. For example, new spectral data for variants of SARS-CoV-2 can be seamlessly integrated to improve detection accuracy and classification precision.

The machine learning module 116 applies classification algorithms, such as Principal Component Analysis (PCA) and Linear Discriminant Analysis (LDA), to compare the captured spectral data against the spectral library. This enables the identification and classification of biological components with high precision, even in complex environmental matrices. The machine learning module 116 also supports updates to the spectral library, allowing the system 102 to adapt to new pathogens as they emerge.

The control unit 118 manages the operations of the system 102, synchronizing the processes performed by the other components. The control unit 118 monitors processing parameters such as temperature, agitation speed, and laser intensity, ensuring optimal performance. The control unit 118 also records operational data and facilitates adjustments to system settings based on user inputs or automated diagnostics.

The power supply unit 120 provides the energy required for system operation. The power supply unit 120 can include a rechargeable battery (e.g., lithium-ion), which supports several hours of continuous operation in the field. A power regulation system ensures consistent voltage and current delivery to all components of the system 102.

The output interface 122 delivers the results of the analysis in real-time and/or can save the results for future review. The output interface 122 may include a display screen or a mobile application that presents qualitative information, such as microorganism identification, and quantitative metrics, such as RNA concentration levels. The output interface 122 can also include one or more connectivity options, such as Wi-Fi, Bluetooth®, Zig-Bee®, Z-Wave*, other short-range connectivity options, other mid-to-long range connectivity options (e.g., cellular-based Internet of Things), radio frequency identifier (RFID), and the like, for transmitting data to external systems. The output 124 can include actionable data that can be used for environmental monitoring, public health planning, or compliance reporting.

FIG. 2 depicts a functional workflow 200 of the system 102, detailing a transformation of a biological sample 104 into actionable output 124 according to an example implementation. The workflow 200 highlights the progression of material and data through the interconnected components of the system 102, starting with the collection of the biological sample 102 and culminating in the generation of analytical results as part of the output 124. Each step in the workflow 200 is described as building upon the output of the preceding unit as managed and synchronized by the control unit 118.

The workflow 200 begins at the environmental source 106, where the biological sample 104 is collected by the sample collection unit 108. For air sampling, the sample collection unit 108 may utilize a filtration system, such as a high-efficiency particulate air (HEPA) filter or cyclone separator, in combination with an integrated pump or fan to draw in and capture airborne particulates, including microorganisms like SARS-CoV-2, Zika, or influenza A. Fluid sampling may involve reservoirs, syringes, or automated pipetting systems, enabling the collection and storage of liquid samples, such as water from treatment facilities, with minimal contamination. For surface sampling, the sample collection unit 108 may use sterile swabs or wipes to collect microbial residues from high-touch surfaces, with the collected material optionally transferred directly into a buffer medium for further processing. The sample collection unit 108 may also include pre-processing features, such as concentration mechanisms (e.g., centrifugation or evaporation) to enhance the abundance of analytes in the sample or sterilization steps to mitigate contamination risks. The processed material, referred to as a pre-processed biological sample 202, is then delivered to the lysis unit 110.

The lysis unit 110 further processes the pre-processed biological sample 202 by breaking down its cellular or viral structures into molecular components. Particularly, the lysis unit 110 may use a chemical, thermal, or mechanical process, or any combination thereof to accelerate a lysis process of the pre-processed biological sample 202. In some implementations, the lysis unit 110 may use a buffer injection system to introduce a Raman-background-free surfactant buffer, such as sodium dodecyl sulfate (SDS), which prevents interference in downstream Raman analysis. Simultaneously, in these implementations, a heating mechanism maintains controlled temperatures (e.g., about 35° C. to 60° C.), and an agitation mechanism (e.g., ultrasonic sonication or mechanical stirring) disrupts cell membranes or viral envelopes. The result of the lysis process is a lysate 204. The lysate 204 is a solution containing the biological sample 104, including one or more structural components decomposed from the lysis process, such as RNA, DNA, lipids, carbohydrates, and proteins. For instance, the lysate 204 from SARS-CoV-2 may include RNA fragments and structural proteins like the spike (S), nucleocapsid (N), and envelope (E) proteins.

The lysate 204 is transferred to the nanoparticle aggregation unit 112, where the lysate 204 is combined with gold, silver, and/or other plasmonic nanoparticles to create an enhanced analyte 206. In the nanoparticle aggregation unit 112 plasmonic nanoparticles, such as gold or silver nanoparticles, may be dispensed from a nanoparticle reservoir into a mixing chamber, whereby the plasmonic nanoparticles interact with the lysate 204. This interaction facilitates electrostatic or chemical bonding between the nanoparticles and the molecular components of the lysate 204. The mixture then may be deposited onto a hydrophobic surface, such as silane-treated aluminum foil, which induces condensation and spatial organization of the components. In addition to silane-treated aluminum foil, the hydrophobic surface may include microfluidic channels or polymer-coated substrates designed to enhance condensation and nanoparticle stability. These configurations enable the system 102 to adapt to different sample types and environmental conditions. This process results in the formation of nanogap plasmonic hotspots, which are regions where the nanoparticles are positioned closely enough to amplify the Raman scattering signals. These hotspots enhance the detectability of molecular vibrations associated with the structural components of the biological sample 104, optimizing the analyte 204 for high-sensitivity Raman spectral analysis in subsequent stages.

The enhanced analyte 206 is analyzed by the spectral analysis unit 114, which uses a laser—typically operating at a wavelength of 785 nanometers—to excite the enhanced analyte 206. The interaction between the laser and the plasmonic hotspots produces Raman scattering signals, which are captured by a detector of the spectral analysis unit 114. The output of this stage is raw spectral data 208 that corresponds to the molecular vibrations of the structural components in the biological sample 104, forming a unique Raman fingerprint.

The raw spectral data 208 is sent to the machine learning module 116 for analysis and classification. The machine learning module 116 may access a SERS spectral library 210, which contains pre-analyzed spectral profiles of known microorganisms, such as SARS-CoV-2, influenza A, and Zika. Using advanced algorithms, such as Principal Component Analysis (PCA) and Linear Discriminant Analysis (LDA), the machine learning module 116 compares the captured spectral data with entries in the SERS spectral library 210 to identify the biological components in the biological sample 104. The machine learning module 116 generates the output 124, which may include qualitative results (e.g., microorganism identification), quantitative metrics (e.g., RNA concentration), or both, which are sent to the output interface 122.

The output interface 122 provides real-time feedback to the user, presenting the results of the analysis in an accessible format. The output 124 may include microorganism identification, concentration levels, and related metrics. Results can be displayed locally on a screen, stored for later retrieval, or transmitted to external systems via connectivity options such as Wi-Fi, Bluetooth®, or cellular networks. This enables integration with centralized monitoring platforms or remote data analysis systems, making the system 102 adaptable to various operational contexts.

Throughout the workflow 200, the control unit 118 manages and synchronizes the components. The control unit 118, for example, may regulate processing parameters, such as temperature and agitation speed in the lysis unit 110, nanoparticle concentration in the nanoparticle aggregation unit 112, and laser intensity in the spectral analysis unit 114. The control unit 118 may also ensure smooth communication between components, monitor system performance, and facilitate user inputs or automated adjustments. The control unit 118 allows the system 102 to adapt dynamically to diverse sample types and environmental conditions, delivering accurate and reliable results across a wide range of applications, including public health monitoring, environmental analysis, and industrial quality control.

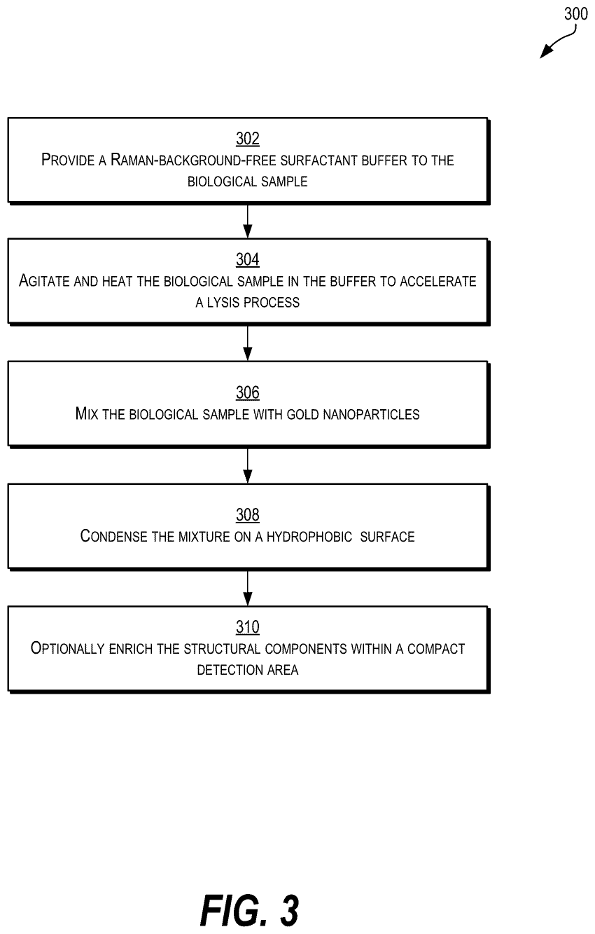

FIG. 3 depicts a method 300 for preparing biological components from a biological sample 104 for label-free SERS according to an example implementation. It should be understood that the operations of the methods described herein are not necessarily presented in any particular order and that performance of some or all of the operations in an alternative order(s) is possible and is contemplated. The operations have been presented in the demonstrated order for ease of description and illustration. Operations may be added, omitted, and/or performed simultaneously, without departing from the scope of the appended claims.

At block 302, the method 300 begins by providing a Raman-background-free surfactant buffer to the biological sample 104. The biological sample 104 may include viruses such as SARS-CoV-2, influenza A, or Zika, as well as bacteria, fungi, or combinations thereof. The surfactant buffer, which may be sodium dodecyl sulfate (SDS) or another Raman-inactive surfactant like Triton X-100, facilitates chemical lysis while ensuring minimal interference with Raman spectral detection. The buffer is introduced in a controlled volume-to-mass ratio to optimize interaction with the sample and to prepare the components for downstream processing.

At block 304, the biological sample 104 is agitated and heated in the surfactant buffer to accelerate a lysis process thereby decomposing structural components of the biological sample. Agitation may involve ultrasonic sonication, mechanical stirring, or acoustic transduction, with ultrasonic sonication at frequencies between 20 kHz and 40 kHz being particularly effective for breaking down cellular membranes and viral envelopes. Heating is controlled within a temperature range of about 35° C. to 60° C., which promotes efficient lysis while preserving the integrity of sensitive analytes such as RNA and proteins. For example, lysing SARS-CoV-2 in this manner ensures the release and preservation of structural proteins such as the spike (S), envelope (E), and nucleocapsid (N) proteins, along with viral RNA fragments critical for downstream analysis.

At block 306, the biological sample is mixed with gold, silver, and/or other nanoparticles to enhance Raman signals through surface plasmon resonance. The gold, silver, and/or other nanoparticles, which may be spherical particles approximately 20-50 nm in diameter, interact chemically or electrostatically with the structural components, forming plasmonic aggregates. These aggregates are critical for amplifying Raman scattering signals by creating nanogap hotspots. For example, RNA and proteins from SARS-CoV-2 may bind to the nanoparticles, facilitating the formation of hotspots for signal enhancement.

At block 308, the mixture of gold, silver, and/or other nanoparticles and the biological sample is condensed onto a hydrophobic surface, such as silane-treated aluminum foil. This step promotes the formation of enriched analytes with nanogap SERS hotspots. The condensation process spatially organizes the nanoparticles and structural components, resulting in highly amplified electromagnetic fields in nanometer-scale gaps between particles. These hotspots significantly improve the sensitivity and accuracy of SERS detection.

At block 310, the method 300 may optionally include an enrichment step to further concentrate the structural components and nanoparticles within a compact detection area. This enrichment increases the density of analytes in the detection zone, optimizing the sample for high-sensitivity Raman spectral analysis. Enrichment may involve controlling the deposition process or using functionalized surfaces that promote nanoparticle aggregation in localized regions. This step ensures that the Raman signal is maximized, even when working with low-concentration samples.

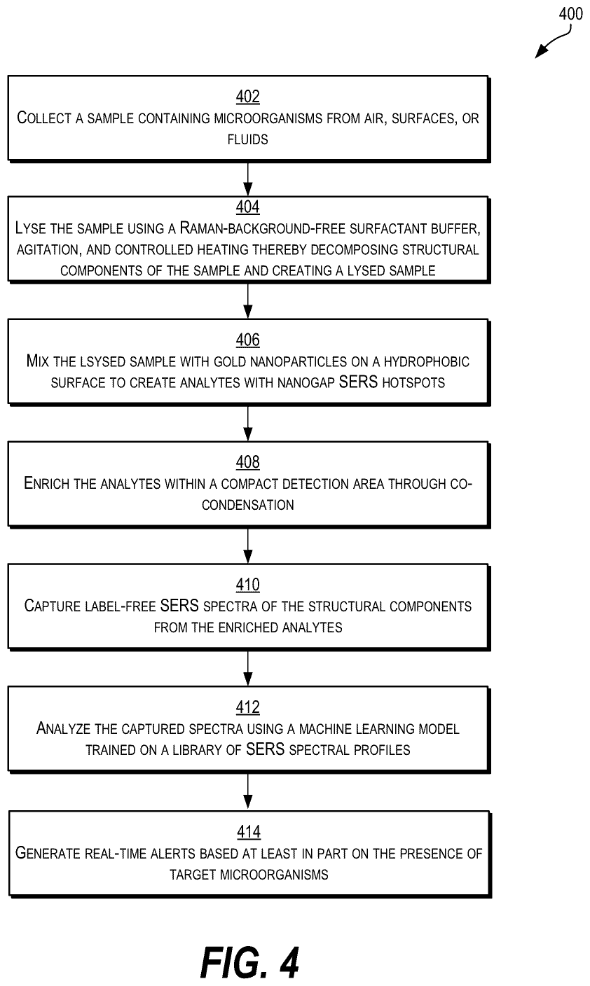

FIG. 4 illustrates a method 400 for continuous, real-time monitoring of microorganisms in environmental samples using label-free SERS according to an example implementation. The method begins at block 402, where a biological sample 104 containing one or more microorganisms is collected from an environmental source 106 such as air, surfaces, or fluids. Airborne microorganisms may be captured using air filters or cyclone separators, while fluid samples, such as drinking water or wastewater, can be collected into reservoirs. Surface residues may be sampled using swabs or wipe mechanisms. The biological sample 104 may include microorganisms such as viruses (e.g., SARS-CoV-2, influenza A, Zika), bacteria, or fungi, depending on the source and application.

At block 404, the collected microorganisms are lysed using a combination of a Raman-background-free surfactant buffer, agitation, and controlled heating thereby decomposing structural components of the biological sample 104 and creating a lysed sample (e.g., the lysate 204). The surfactant buffer, which may be sodium dodecyl sulfate (SDS) or another Raman-inactive surfactant, ensures chemical lysis without introducing interfering Raman spectral peaks. The agitation may be achieved through ultrasonic sonication, mechanical stirring, or acoustic transduction, with sonication frequencies between 20 kHz and 40 kHz being particularly effective for breaking down microbial structures. Controlled heating, typically maintained within a range of 35° C. to 60° C., enhances lysis efficiency while preserving the integrity of sensitive analytes such as RNA and proteins. For example, this step can release viral RNA and structural proteins, including the spike (S), envelope (E), and nucleocapsid (N) proteins of SARS-CoV-2.

At block 406, the lysed sample is mixed with gold, silver, and/or other nanoparticles on a hydrophobic surface. The gold, silver, and/or other nanoparticles interact chemically or electrostatically with the structural components, forming plasmonic aggregates with nanogap SERS hotspots. These hotspots amplify the Raman signals generated during subsequent spectral analysis. A hydrophobic surface, such as silane-treated aluminum foil, is used to promote the spatial organization and condensation of the structural components and nanoparticles, ensuring uniform distribution and stable interactions.

At block 408, the analytes are enriched within a compact detection area through a co-condensation process. This step involves concentrating the structural components and nanoparticles into a confined region on the hydrophobic surface, maximizing the analyte density and optimizing the sample for Raman spectral detection. The co-condensation process may be performed at a temperature range of 20° C. to 30° C., which preserves the structural integrity of biomolecules such as RNA and proteins while ensuring effective condensation.

At block 410, the enriched analytes are subjected to Raman spectroscopy to capture label-free SERS spectra. A laser, typically operating at a wavelength of 785 nanometers, excites the analytes, producing Raman scattering signals that correspond to the molecular vibrations of the structural components. These signals are detected and recorded, generating unique spectral profiles that represent the molecular fingerprints of the analytes. For example, the Raman spectra may capture the vibrational modes of RNA nucleotide bases or the amino acid compositions of proteins associated with specific microorganisms.

At block 412, the captured SERS spectra are analyzed using a machine learning model trained on a library of SERS spectral profiles. This spectral library may include data for RNA nucleotide bases and protein amino acid compositions associated with microorganisms, such as SARS-CoV-2, influenza A, and Zika. The machine learning model applies classification algorithms, such as Principal Component Analysis (PCA) or Linear Discriminant Analysis (LDA), to compare the captured spectra against the library, enabling precise identification and characterization of the microorganisms present in the sample.

At block 414, the output interface 122 generates real-time alerts based at least in part on the detection of target microorganisms. These alerts, which may be delivered through a system interface or transmitted to external devices, provide actionable information for applications such as environmental monitoring, pathogen containment, or public health surveillance. For example, the detection of SARS-CoV-2 in an air sample could trigger an immediate alert for facility managers or health authorities.

The present disclosure is directed to the detection and analysis of microorganisms in environmental samples using label-free SERS. The disclosed approach integrates several innovative features, including the use of a Raman-background-free surfactant buffer to eliminate spectral interference, the application of advanced lysis techniques to efficiently release structural components, and the incorporation of gold, silver, and/or other nanoparticles to create nanogap SERS hotspots for signal amplification. Further, the co-condensation of analytes onto a hydrophobic surface enhances detection sensitivity by enriching the structural components within a compact detection area.

The present disclosure also utilizes machine learning models trained on comprehensive libraries of SERS spectral profiles, enabling the accurate identification of microorganisms, including viruses such as SARS-CoV-2, influenza A, and Zika, as well as bacteria and fungi. The ability of the disclosed system to generate real-time alerts based on microorganism detection provides actionable insights for public health monitoring, environmental analysis, and industrial quality control. By combining rapid sample preparation, enhanced analyte detection, and intelligent data analysis in a compact and portable format, the present disclosure offers a significant improvement over traditional methods, enabling high-sensitivity, continuous monitoring of microorganisms in diverse environments.

The features, structures, or characteristics described above may be combined in one or more implementations in any suitable manner, and the features discussed in the various implementations are interchangeable, if possible. In the following description, numerous specific details are provided in order to fully understand the embodiments of the present disclosure. However, a person skilled in the art will appreciate that the technical solution of the present disclosure may be practiced without one or more of the specific details, or other methods, components, materials, and the like may be employed. In other instances, well-known structures, materials, or operations are not shown or described in detail to avoid obscuring aspects of the present disclosure.

In this specification, the terms such as “a,” “an,” “the,” and “said” are used to indicate the presence of one or more elements and components. The terms “comprise,” “include,” “have,” “contain,” and their variants are used to be open ended, and are meant to include additional elements, components, etc., in addition to the listed elements, components, etc. unless otherwise specified in the appended claims.

The terms “first,” “second,” etc. are used only as labels, rather than a limitation for a number of the objects. It is understood that if multiple components are shown, the components may be referred to as a “first” component, a “second” component, and so forth, to the extent applicable.

The above-described implementations of the present disclosure are merely possible examples set forth for a clear understanding of the principles of the disclosure. Many variations and modifications may be made to the above-described implementations without departing substantially from the spirit and principles of the disclosure. All such modifications and variations are intended to be included herein within the scope of this disclosure and protected by the following claims.

What is claimed is:

1. A method for preparing biological components from a biological sample for label-free surface-enhanced Raman spectroscopy (SERS), comprising:
providing a Raman-background-free surfactant buffer to the biological sample; and
agitating and heating the biological sample in the buffer to accelerate a lysis process thereby decomposing structural components of the biological sample, and wherein the structural components are substantially free of Raman background interference and are optimized for SERS signal enhancement.

2. The method of claim 1, further comprising:
mixing the structural components with nanoparticles to form a mixture; and
condensing the mixture onto a hydrophobic surface to form enriched analytes with nanogap SERS hotspots.

3. The method of claim 1, wherein the biological sample comprises a virus, a bacterium, a fungus, or a combination thereof.

4. The method of claim 1, wherein the structural components comprise at least one of a protein, a lipid, a carbohydrate, or a nucleic acid.

5. The method of claim 1, wherein the surfactant buffer comprises sodium dodecyl sulfate (SDS) or another Raman-inactive buffer.

6. The method of claim 1, wherein the biological sample is heated to a temperature between about 35° C. and 60° C. to optimize lysis efficiency while preserving integrity of the structural components.

7. The method of claim 1, wherein the agitation is performed using ultrasonic sonication at a frequency between 20 kHz and 40 kHz, mechanical agitation, or acoustic transduction.

8. The method of claim 1, further comprising enriching the structural components within a compact detection area by co-condensing the lysed sample and nanoparticles on a silane-functionalized hydrophobic surface.

9. A method for continuous, real-time monitoring of microorganisms in environmental samples, comprising:
collecting a sample containing microorganisms from air, surfaces, or fluids;
lysing the sample using a Raman-background-free surfactant buffer, agitation, and controlled heating thereby decomposing structural components of the sample and creating a lysed sample;
mixing the lysed sample with gold or silver nanoparticles on a hydrophobic surface to create analytes with nanogap surface-enhanced Raman spectroscopy (SERS) hotspots;
enriching the analytes within a compact detection area through co-condensation;
capturing label-free SERS spectra of the structural components from the enriched analytes; and
analyzing the captured spectra using a machine learning model trained on a library of SERS spectral profiles.

10. The method of claim 9, wherein the machine learning model is trained on a library comprising spectral profiles of macromolecular components associated with microorganisms.

11. The method of claim 9, wherein the library comprises SERS data for structural components of SARS-CoV-2, influenza A virus, and Zika virus.

12. The method of claim 9, wherein the environmental sample is collected from an air filter, a high-touch surface, or a fluid sample.

13. The method of claim 9, further comprising generating real-time alerts based at least in part on a detected presence of a target microorganism.

14. The method of claim 9, wherein the hydrophobic surface comprises silane-treated aluminum foil.

15. The method of claim 9, wherein the co-condensation is performed at a temperature between about 20° C. and 30° C. to preserve a structural integrity of the sample.

16. A portable system for label-free detection of biological components in environmental samples, comprising:
a lysis unit configured to process biological samples using a surfactant buffer, agitation, and controlled heating to form a lysate;
a nanoparticle aggregation module configured to mix the lysate with gold or silver nanoparticles to form a mixture and condense the mixture onto a hydrophobic surface to create analytes with nanogap surface-enhanced Raman spectroscopy (SERS) hotspots;
a Raman spectrometer configured to capture spectral data from the analytes; and
a machine learning module trained with a library of SERS spectral profiles for real-time classification of microorganisms.

17. The system of claim 16, wherein the lysis unit comprises an ultrasonic sonicator capable of operating at frequencies between 20 kHz and 40 kHz, a mechanical agitator, or an acoustic transducer.

18. The system of claim 16, wherein the machine learning module is configured to identify microorganisms based on spectral differences in ribonucleic acid (RNA) nucleotide and protein peak compositions.

19. The system of claim 16, wherein the hydrophobic surface comprises a microfluidic channel coated with silane-treated materials.

20. The system of claim 16, wherein the Raman spectrometer is a compact, portable device optimized for field deployment.
